(19) United States
(12) Patent Application Publication
(10) Pub. No.: US 2007/0043428 A1
(43) Pub. Date: Feb. 22, 2007

(54) Barrier stent and use thereof

(75) Inventors: Lisa K. Jennings, Memphis, TN (US); Chunxiang Zhang, Memphis, TN (US); Larry C. Wadsworth, Knoxville, TN (US); Randall R. Bresee, Walland, TX (US); Roberto S. Benson, Knoxville, TN (US); Christopher P. Stephens, Knoxville, TN (US)

Correspondence Address: NIXON PEABODY LLP - PATENT GROUP, CLINTON SQUARE, P.O. BOX 31051, ROCHESTER, NY, 14603-1051, US

(73) Assignee: The University of Tennessee Research Foundation, Knoxville, TN (US)

(21) Appl. No.: 11/371,652

(22) Filed: Mar. 9, 2006

Related U.S. Application Data
(60) Provisional application No. 60/659,899, filed on Mar. 9, 2005.

(51) Int. Cl. A61F 2/06

(52) U.S. Cl. 623/1.15; 623/1.42; 427/2.25

(57) ABSTRACT

The present invention relates to a vascular stent that includes an expandable stent defining an interior compartment, a first polymeric layer exposed to the interior compartment defined by the stent, the first layer comprising an agent that promotes re-endothelialization, an agent that inhibits thrombosis, or a combination thereof, and a second polymeric layer at least partially external of the stent, the second layer being adapted for contacting a vascular surface and being characterized by pores that are substantially impermeable to vascular smooth muscle cell migration. Method of making and using the vascular stent are also disclosed.

This application claims the benefit of U.S. Provisional Patent Application Ser. No. 60/659,899, filed Mar. 9, 2005, which is hereby incorporated by reference in its entirety.

### FIELD OF THE INVENTION

The present invention relates generally to a novel stent construction; use thereof to prevent thrombosis and neointima formation, and thereby treat coronary or vascular diseases; as well as methods of manufacture.

### BACKGROUND OF THE INVENTION

More than 1.5 million patients receive percutaneous transluminal coronary angioplasty (“PTCA”) and peripheral artery angioplasty (“PTA”) every year in the world. Despite being successful procedures, PTCA and PTA remain limited by restenosis that occurs in 30-60% of patients (Rajagopal et al., Am. J Med. 115:547-553 (2003)). Thus, restenosis after angioplasty is not only important clinically but also for its impact on health-care costs.

The pathological mechanisms of restenosis are neointimal formation, elastic recoil, and vascular negative remodeling (Isner, Circulation 89:2937-2941 (1994); Mintz, Curr. Interv. Cardiol. Rep. 2(4):316-325 (2000); Schwartz et al., Rev. Cardiovasc. Med. 3 Suppl 5:S4-9 (2002)). Both elastic recoil and negative remodeling have been successfully addressed to a large extent by the development of endovascular stents. Indeed, clinical trials have established stents as the first mechanical intervention to have a favorable impact on restenosis (Rajagopal et al., Am. J. Med. 115:547-553 (2003); Bittl et al., Am. J. Cardiology 70:1533-1539 (1992); Fischman et al., Radiology 148: 699-702 (1983)). Although, the conventional endovascular stents are able to block elastic recoil and vascular negative remodeling, resulting in the reduction of the restenosis rate by about 10%, they cannot inhibit neointima thickening, and may even increase neointima formation which results in in-stent restenosis (Bennett, Heart 89(2):218-224 (2003); Holmes, Jr., Rev. Cardiovasc. Med. 2(3):115-119 (2001); Lowe et al., J. Am. Coll. Cardiol. 39(2):183-193 (2002); Virmani et al., Curr. Opin. Lipidol. 10(6):499-506 (1999); Hanke et al., Herz. 17(5):300-308 (1992)). Therefore, although the advent of endovascular stents has reduced the incidence of restenosis, the problem still occurs in 20-30% of stented vessels (Rajagopal et al., Am. J Med. 115:547-553 (2003)).

Neointimal formation, the result of complex multi-cellular events and the most important and final cellular event responsible for neointima thickening, is a consequence of vascular smooth muscle cell proliferation and migration (Steele et al., Circ. Res. 57:105-112 (1985); Teirstein et al., Circulation 101 :360-365 (2000); Pauletto et al., Clin. Sci. 87(5):467-479 (1994); Bauters et al., Prog. Cardiovasc. Dis. 40(2):107-116 (1997); Hanke et al., Eur. Heart J 16(6):785-793 (1995); Kocher et al., Lab. Invest. 65:459-470 (1991)). Balloon injury (i.e., from the angioplasty) causes damage to vascular endothelial cells. Preceding neointimal formation is activation of smooth muscle cells in the injured media by the response from the vascular wall and the numerous pro-proliferative factors in blood (Regan et al., J. Clin. Invest. 106(9):1139-1147 (2000); Aikawa et al., Circulation 96(1):82-90 (1997); Ueda et al., Coron. Artery Dis. 6(1):71-81 (1995); Hanke et al., Circ. Res. 67(3):651-659 (1990)). The initial activation response is followed by proliferation and migration of vascular smooth muscle cells into the intima (Pauletto et al., Clin. Sci. 87(5):467-479 (1994); Bauters et al., Prog. Cardiovasc. Dis. 40(2):107-116 (1997); Hanke et al., Eur. Heart J. 16(6):785-793 (1995); Kocher et al., Lab. Invest. 65:459-470 (1991); Garas et al., Pharmacol. Ther. 92(2-3):165-178 (2001)). Under stented conditions, the VSMC are able to migrate into the inside of the stent through the mesh (Bennett, Heart 89(2):218-224 (2003); Holmes, Jr., Rev. Cardiovasc. Med. 2(3): 115-119 (2001); Lowe et al., J. Am. Coll. Cardiol. 39(2):183-193 (2002); Virmani et al., Curr. Opin. Lipidol. 10(6):499-506 (1999); Hanke et al., Herz. 17(5):300-308 (1992)). The VSMC in intima will multiply and synthesize an extracellular matrix resulting in the neointima formation and restenosis (Hanke et al., Herz. 17(5):300-308 (1992); Pauletto et al., Clin. Sci. 87(5):467-479 (1994); Bauters et al., Prog. Cardiovasc. Dis. 40(2):107-116 (1997); Hanke et al., Eur. Heart J. 16(6):785-793 (1995); Kocher et al., Lab. Invest. 65:459-470 (1991); Garas et al., Pharmacol. Ther. 92(2-3):165-178 (2001)). The critical role of VSMC proliferation in the development of atherosclerosis has been confirmed by numerous basic and clinical studies, in which anti-proliferation of VSMC either by systemic approach or local delivery approach successfully reduces restenosis (Kuchulakanti et al., Drugs 64(21):2379-2388 (2004); Andres et al., Curr. Vasc. Pharmacol. 1(l):85-98 (2003); Fattori et al., Lancet 361(9353):247-249 (2003); Cutlip, J. Thromb. Thrombolysis 10(1):89-101 (2000)).

Events related to thrombosis, such as platelet activation, platelet deposition, overexpression of tissue factor, and mural thrombus at sites of vascular injury, are the early responses to vascular balloon injury and to stent implantation (Chandrasekar et al., J. Am. Coll. Cardiol. 35(3):555-562 (2000); Conde et al., Catheter Cardiovasc. Interv. 60(2):236-246 (2003); Ischinger, Am. J Cardiol. 82(5B):25L-28L (1998); Clowes et al., Lab Invest. 39:141-150 (1978)). It is clear that platelets, by their capacity to adhere to the sites of arterial injury, form aggregates, and secrete highly potent growth factors, appear to play an important role in VSMC proliferation and development of restenosis. Many novel drugs and delivery systems that target platelets and thrombosis reduce restenosis both in animals and in humans (Ischinger, Am. J. Cardiol. 82(5B):25L-28L (1998); Clowes et al., Lab Invest. 39:141-150 (1978)). A novel candidate for inhibiting arterial thrombosis is GPVI, a platelet specific cell surface receptor responsible for platelet adhesion and activation to collagen. It is now accepted that GPVI is the principle receptor for collagen-induced platelet activation, and is a critical conduit for signal transduction (Ichinohe et al., J. Biol. Chem. 270(47):28029-28036 (1995); Tsuji et al., J. Biol Chem. 272(28):23528-23531 (1997)). In contrast, the other major collagen receptor in platelets, GPIa-IIa, is primarily involved with the cation-dependent processes of spreading and cell-cell cohesion.

The physiological functions of the vascular endothelial cell endothelium include: barrier regulation of permeability, thrombogenicity, and leukocyte adherence, as well as production of growth-inhibitory molecules. These molecules are critical to the prevention of luminal narrowing by neointimal thickening. Therefore, an intact endothelium appears to be nature's means of preventing intimal lesion formation. However, after angioplasty and stent implantation, the endothelial cells are damaged and/or denuded. An inverse relationship between endothelial integrity and VSMC proliferation has been well established in animal models (Bjorkerud et al., Atherosclerosis 18:235-255 (1973); Fishman et al., Lab Invest. 32:339-351 (1975); Haudenschild et al., Lab Invest. 41:407-418 (1979); Davies et al., Br. Heart J. 60:459-464 (1988)). Data regarding the relationship between endothelial integrity and neointirnal thickening in human arteries, though limited, are consistent with the results of animal experiments (Schwarcz et al., J. Vasc Surg. 5:280-288 (1987); Gravanis et al., Circulation 107(21):2635-2637 (2003); Kipshidze et al., J. Am. Coll. Cardiol. 44(4):733-739 (2004)).

Acceleration of re-endothelialization either by drugs or by endothelial cell seeding is reported to reduce neointima growth after angioplasty and stent implantation (Walter et al., Circulation 110(l):36-45 (2004); Chuter, Cardiovasc. Surg. 10(1):7-13 (2002); Conte et al., Cardiovasc. Res. 53(2):502-511 (2002); Garas et al., Pharmacol. Ther. 92(2-3):165-178 (2001); Edelman et al., Am. J Cardiol. 81, pp. 4E-6E (1998)).

The first attempts to stop restenosis employed radiation. A gamma or beta source was applied to a ribbon left in the lesion temporarily after stenting or incorporated into stent material (Schwartz et al., Rev. Cardiovasc. Med. 3 Suppl 5:S4-9 (2002)). Such irradiation does indeed inhibit neointima formation (Mintz, Curr. Interv. Cardiol. Rep. 2(4):316-325 (2000); Bittl et al., Am. J. Cardiology 70:1533-1539 (1992)), but intravascular brachytherapy has two undesirable consequences: an increase in the risk of thrombosis and stimulation of hyperplasia at the ends of the stent (the candy wrapper effect). The U.S. Food and Drug Administration (“FDA”), therefore, has approved such devices only for the treatment of in-stent restenosis, not for primary stenting.

Current attention is now focused on antiproliferative drugs that are delivered locally, via polymer coatings that surround the bare-metal stents (i.e., coated stents). There are currently on the market two widely-used coated stents. The first is a balloon-expandable stainless-steel stent carrying sirolimus in a two-polymer coating; this was approved by the FDA in April 2003. The Health Alliance of Greater Cincinnati has estimated that 10% of bypass operations will be replaced by insertion of the drug eluting stents, 15% of straightforward angioplasty procedures will change to stenting, and that use of the coated stents would reduce re-admissions by 25%.

The current popularity of radioactive and drug-eluting stents is due in large part to the fact that they are much more effective in inhibiting early neointimal growth compared to bare-metal stents (Leon et al., N. Engl. J. Med. 344:250-256 (2001); Liistro et al., Circulation 105:1883-1886 (2002); Kolodgie et al., Circulation 106:1195-1198 (2002); Morice et al., N. Engl. J. Med. 346:1773-1780 (2002); Waksman et al., J. Am. Coll. Cardiol. 36:65-68 (2000)). In both cases, the strategy of targeting proliferating VSMC at the site of injury has been successful in reducing neointimal lesion formation. The early intriguing success of these interventions, however, has exposed a potential liability of an indiscriminate antiproliferative approach for restenosis prevention. Indeed, the delayed re-endothelialization and the incidence of late thrombosis (Farb et al., Circulation 103:1912-1919 (2001); Liistro et al., Heart 86:262-264 (2001); Guba et al., Nat. Med. 8:128-135 (2002); Asahara et al., Circulation 91(11):2793-801 (1995)), due to nonselective growth inhibition of VSMC and endothelial cells, were found in both radioactive and drug-eluting stents. Therefore, such an approach may only delay the proliferative responses rather than prevent them and the long-term consequences remain to be defined at this time (Farb et al., Circulation 103:1912-1919 (2001); Liistro et al., Heart 86:262-264 (2001); Guba et al., Nat. Med. 8:128-135 (2002); Asahara et al., Circulation 91(11):2793-801 (1995)).

The use of non-porous external coatings on stents has been described previously (Marin et al., J. Vasc. Interv. Radiol. 7(5):651-656 (1996); Yuan et al., J. Endovasc. Surg. 5(4):349-358 (1998)), but these coatings did not provide for endothelial cell migration, nor were they utilized in combination with other materials.

Although stent grafts which are currently used for arterial aneurysms also have a cover on the outside surface of the stent, the cover is made of multi-porous material that is cell permeable (Palmaz et al., J. Vasc. Interv. Radiol. 7(5):657-63 (1996); Zhang et al., Biomaterials 25(1):177-87 (2004); Indolfi et al., Trends Cardiovasc. Med. 13(4):142-8 (2003)). VSMC in the vascular wall are therefore able to migrate toward the lumen through the pores of these covers. Currently, covered stents have no inner layer for acceleration of re-endothelialization.

Thus, there still remains a need for a vascular stent that can promote early re-endothelialization while preventing in-stent neointima and thromosis. The present invention is directed to overcoming these and other deficiencies in the art.

### SUMMARY OF THE INVENTION

A first aspect of the present invention relates to a vascular stent that includes: an expandable stent defining an interior compartment; a first polymeric layer exposed to the interior compartment defined by the stent, the first layer including an agent that promotes re-endothelialization, an agent that inhibits thrombosis, or a combination thereof; and a second polymeric layer at least partially external of the stent, the second layer being adapted for contacting a vascular surface and being characterized by pores that are substantially impermeable to vascular smooth muscle cell (“VSMC”) migration. According to one preferred embodiment, the second layer has pores that are substantially impermeable to all cells. According to another preferred embodiment, the second layer has pores that are permeable to squamous epithelial cells or endothelial cells but not the VSMC.

A second aspect of the present invention relates to a method of preventing neointimal hyperplasia in a patient following insertion of a prosthetic graft. This method involves providing a vascular stent according to the first aspect of the present invention; and inserting the vascular stent at a vascular site of the patient, wherein the material of the second polymeric layer substantially precludes migration of vascular smooth muscle cells internally of stent and thereby prevents neointimal hyperplasia.

A third aspect of the present invention relates to a method of preventing in-stent thrombosis. This method involves providing a vascular stent according to the first aspect of the present invention, wherein the first polymeric layer comprises an agent that inhibits thrombosis; and inserting the vascular stent at a vascular site of the patient, wherein release of the agent that inhibits thrombosis from the first polymeric layer substantially precludes aggregation of platelets (i.e., in-stent) and thereby prevents in-stent thrombosis.

A fourth aspect of the present invention relates to a method of treating a coronary artery disease, peripheral artery disease, stroke, or other vascular bed disease. This method involves the steps of providing a vascular stent according to the first aspect of the present invention; performing angioplasty at a vascular site in a patient exhibiting conditions associated with coronary artery disease, peripheral artery disease, or stroke; inserting the vascular stent at the vascular site, wherein said inserting substantially precludes neointima and in-stent thrombosis while promoting re-endothelialization, thereby treating coronary artery disease, peripheral artery disease, stroke, or other vascular bed disease.

A fifth aspect of the present invention relates to a method of making a vascular stent of the present invention. This method is carried out by providing an expandable stent that defines an interior compartment; applying to at least an internal surface of the expandable stent a first polymeric material comprising an agent that promotes re-endothelializafion, an agent that inhibits thrombosis, or a combination thereof, thereby forming the first polymeric layer exposed to the interior compartment; and covering at least an outer surface of the expandable stent with a second polymeric material in a manner that maintains stent expandability and forms a porous second polymeric layer having pores that are substantially impermeable to vascular smooth muscle cell migration.

The vascular stents of the present invention are preferably characterized by an outer coating that contains pores engineered to be intermediate between the coarse open structure of conventional bare metal stents, which allow penetration of nearly all substances, and a solid barrier which blocks penetration of nearly all substances. According to one embodiment, the outer coating is an elastic film or elastic fibrous (i.e., woven or non-woven) coating that allows for small molecule permeability, like water and proteins, but blocks the penetration of all cells. According to a second embodiment, the outer coating is a web of elastic fibers with pores that have high aspect ratios and widths in the range of a several micrometers. As a consequence, the outer coating is sufficiently porous to encourage preferential penetration of squamous epithelial cells. In addition to the outer coating, the vascular stents of the present invention include one or more drug delivery layers. According to one embodiment, drug delivery is produced by a composite of materials that release different drugs at different rates. In addition to its unique mechanism to inhibit neointima formation, this novel stent maintains the benefits of current drug-coated stents.

### BRIEF DESCRIPTION OF THE DRAWINGS

FIG. 1A is a perspective view of one embodiment of a vascular stent of the present invention inserted within a vessel. The enlarged cross-sectional view (FIG. 1B) illustrates the inner and outer coatings of the stent.

FIG. 2 schematically illustrates a drug-eluting polymer coating as used on the vascular stent of the present invention.

FIG. 3 is a graph illustrating the expected drug release profile resulting from the combination of a fast-release film (e.g., polyurethane-polyethylene glycol) in combination with a slow-release, core-shell bi-component fiber. Drugs can also be grafted onto the films to provide a steady rate of diffusion.

FIG. 4 is a graph illustrating the luminal areas inside stents 14 and 28 days post-angioplasty, comparing the results achieved with a conventional stent (control) lacking an outer barrier to a stent possessing an impermeable outer polyethylene barrier (new).

FIG. 5 is a graph illustrating the neointimal areas within the control and new stents 14 and 28 days post-angioplasty.

FIGS. 6A-B are cross-sectional photomicrograph images illustrating neointima formation and luminal area of rat carotid artert 14 and 28 days post-angioplasty using control or new stents. Tissues were hematoxylin-eosin stained. Original magnification was 4× in FIG. 6A and 10× in FIG. 6B.

FIG. 7 is an SEM photomicrograph of electrospun polyurethane nanofibers.

### DETAILED DESCRIPTION OF THE INVENTION

The present invention relates to an improved vascular stent and the use thereof. The vascular stents of the present invention are designed to: (i) block elastic recoil, (ii) promote re-endothelialization of the vascular site into which the stent was inserted by inhibiting vascular smooth muscle cell infiltration into the interior compartment of the stent while at the same time promoting squamous epithelial or endothelial cell proliferation and migration into the interior compartment; and (iii) inhibit in-stent thrombosis.

The vascular stents of the present invention are formed using an expandable stent. The expandable stent can have any suitable construction, but preferably has a mesh construction that allows for in situ expansion of the stent by any suitable means (e.g., balloon expansion). Suitable stent materials include, among others, metals and monofilament polymeric materials. Exemplary metals include, without limitation, nitinol, gold, platinum, stainless steel, tantalum alloy, cobalt chrome alloy, platinum/tungsten alloy, etc. Exemplary monofilament polymeric materials include, without limitation, polyurethane, polyetherester, ethylene copolymers (e.g., ethylene and vinyl acetate (EVA), ethylene and methylacrylate (E-MA), etc.), polyesters, copolyesters, polyamides, polypropylene, and polyethylene.

The expandable stent defines an interior compartment and includes an inner surface and an outer surface. At least the inner surface is coated with a first polymeric layer that is exposed to the interior compartment defined by the stent, and at least the outer surface is coated with a second polymeric layer. The first layer can be continuous (e.g., a woven or non-woven sheet or a film covering the entire inner surface) or discontinuous (e.g., merely a coating of the stent mesh). According to one embodiment, the second polymeric layer is entirely external of the mesh structure of the stent. According to another embodiment, the second polymeric layer penetrates at least partially within the mesh structure of the stent. The first and second layers are each preferably biocompatible, bioadsorbable, and/or biodegradable.

The first polymeric layer can serve up to two functions: one as a drug delivery vehicle, and the other as a material that promotes in-stent re-endothelialization. Suitable materials that both promote in-stent re-endothelialization and can be used to delivery drugs include, without limitation, hydrogels, porous polyurethane, polytetrafluoroethylene (PTFE), poly(ethylene terephthalate) (PET), aliphatic polyoxaesters, polylactides (PLA), polyglycolide (PGA), polycaprolactones, and combinations thereof. This polymeric layer can include any further additives to enhance its drug delivery and/or re-endothelialization properties.

Exemplary hydrogels include, without limitation, alginate, carageenan, agarose, polyalkylene glycol (e.g., polyethylene glycol), polyvinyl alcohol, polyvinyl acetate, polyvinylpyrrolidone, polyacrylamide, polyacrylic acid, polyhydroxyalky (meth)acrylates, polyalkylene oxides, polyglycolic acids, polylactic acid, and polyglycolic acid-polylactic acid copolymers.

The first layer can also include an agent that promotes re-endothelialization, an agent that inhibits thrombosis, or a combination thereof.

The first polymeric layer is preferably between about 0.5 μm to about 100 μm thick, more preferably between about 5 μm to about 50 μm thick. When used as a drug delivery vehicle, the first polymeric layer preferably is used for rapid drug release, being able to deliver the drug for up to about 30 days.

The first polymeric layer can either coat primarily the interior surface of the stent mesh, or alternatively the first polymeric layer can coat the entire stent (i.e., by dip coating as described hereinafter).

The second polymeric layer preferably serves two functions: one as a drug delivery vehicle and the other as a barrier against vascular smooth muscle cell (“VSMC”) migration. Regardless of the physical position of the second polymeric layer (as identified above), the second polymeric layer is adapted for contacting a vascular surface and is characterized by pores that are substantially impermeable to VSMC migration.

The second polymeric layer is preferably between about 0.05 to about 0.5 mm thick, more preferably 0.1 to about 0.3 mm thick.

According to one embodiment, the second polymeric layer has pores that are substantially impermeable to all cells. In this embodiment, water, small molecules, and proteins can pass through the second polymeric layer. In this embodiment, the average pore width is between about 100 nm to about 5 μm, more preferably between about 200 nm to about 4 μm, or even about 250 nm up to about 2 μm. In this embodiment, the pore shape is preferably substantially elongated with an aspect ratio between about 1.5 and about 20, more preferably between about 2.5 and about 15. Pore aspect ratio is the pore length divided by the pore width.

According to a another embodiment, the second polymeric layer has pores that are permeable to squamous epithelial cells or endothelial cells but not the VSMC. VSMC are typically about 80 to 150 microns in diameter and about 8 microns wide, whereas endothelial cells are typically about 20 to 110 microns in diameter and about 7 microns wide (Haas et al., Microvasc Res. 53(2):113-120 (1997), which is hereby incorporated by reference in its entirety). The size of mobile endothelial cell or VSMC will vary slightly from these ranges due to cytoskeletal restructuring. In accordance with this embodiment, the average pore width of the second polymeric layer is between about 5 μm to about 15 μm, more preferably between about 5 μm to about 10 μm, most preferably between about 5 μm to about 7.5 μm. In this embodiment, the pore shape is preferably substantially elongated with an aspect ratio between about 1.5 and about 20, more preferably between about 2.5 and about 15.

Any suitable material or construction of the second polymeric layer can be utilized to achieve the desired effect. Exemplary polymers or co-polymers include, without limitation, polyurethanes, poly(ethylene oxides), polycarbonates, polystyrenes, polyacrylonitriles, polyamides, polyetheresters (e.g., Domique®), ethylene copolymers (e.g., EVA, E-MA, etc.). The polymeric layer can include any further additives to enhance pore structure or drug delivery properties. Exemplary additives include polyethylene glycol (PEG), and poly(vinyl alcohol) (PVA).

Exemplary agents that promote re-endothelialization include, without limitation, vascular endothelial growth factor (VEGF) and active fragments thereof, angiopoietin-1 and active fragments thereof, and αvβ3 agonists. VEGF is preferred because it is a maintenance and protection factor for endothelial cells as well as a permeability, proliferatory, and migratory factor (Walter et al., Circulation 1 10(1):36-45 (2004); Chuter, Cardiovasc. Surg. 10(1):7-13 (2002), each of which is hereby incorporated by reference in its entirety). Angiopoietin-1 is preferred because it has been shown to be an endothelial specific growth factor (Kanda et al., Cancer Res. 65(15):6820-6827 (2005); Koh et al., Exp. Mol. Med. 34(1):1-11 (2002), each of which is hereby incorporated by reference in its entirety).

Exemplary agents that inhibit thrombosis include, without limitation, GPVI antagonists (including monoclonal anti GPVI antibodies and active single-chain fragments thereof such as Fab fragments), antagonists to the platelet adhesion receptor, (GP1b-V-IX) or to the platelet aggregation receptor (GPIIb-IIIa), both of which can be monoclonal or polyclonal antibodies or fragments thereof (Zhang et al., J. Lab. Clin. Med. 140(2):119-125 (2002), which is hereby incorporated by reference in its entirety), an anti-thrombin antibody, activated protein C (Lin et al., J. Vasc. Interv. Radiol. 14(5):603-611 (2003), which is hereby incorporated by reference in its entirety), heparin, Syk inhibitors such as piceatannol and oxindole (Lai et al., Bioorg Med Chem Lett. 13:3111-3114 (2003), which is hereby incorporated by reference in its entirety), P13-K p110 beta isoform (Jackson et al., Nature Med. 6:507-514 (2005), which is hereby incorporated by reference in its entirety), CD40L antagonists (including anti-CD40L antibodies and fragments thereof) (Prasad et al., Proc. Natl. Acad. Sci. USA 100(21):12367-12371 (2003); Nakamura et al., Rheumatology 45(2):150-156 (2006); Tanne et al., Int. J. Cardiol. 107(3):322-326 (2006), each of which is hereby incorporated by reference in its entirety). Of these, GPVI antagonists and Syk inhibitors are preferred.

Assays to identify other GPVI antagonists include the constant flow assay similar to that described in Moroi et al., Blood 88(6):2081-2092 (1996), which is hereby incorporated by reference in its entirety, or in the plate assay described in Matsuno et al., Br. J Haematol. 92:960-967 (1996), which is hereby incorporated by reference in its entirety, and in Nakamura et al., J. Biol. Chem. 273(8):4338-4344 (1998), which is hereby incorporated by reference in its entirety. In each case, candidate GPVI antagonists can be pre- or co- incubated with the reaction components in the presence and absence of Mg²⁺. Incubation in the absence of Mg²⁺ (e.g., in the divalent cation-free adhesion buffer) blocks the function of GPIa/IIa such that the remaining collagen-dependent activity is primarily mediated by the GPVI receptor.

In addition to the above-identified first and second polymeric layers, the vascular stent of the present invention can also include one or more additional polymeric layers that function primarily as drug delivery vehicles. The one or more additional polymeric layers preferably have different delivery rates from the first and second polymeric layers. The drug(s) to be delivered by the one or more additional polymeric layers can be the same or different from the agent that promotes re-endothelialization and/or the agent that inhibits thrombosis.

Additional drugs that can be delivered via the one or more additional polymeric layers include, without limitation, basic fibroblast growth factor (bFGF) and active fragments thereof, rapamycin and rapamycin analogs, paclitaxel (Taxol™) or Taxan™, antisense dexamethasone, angiopeptin, Batimistat™, Translast™, Halofuginon™, nicotine, acetylsalicylic acid (ASA), Tranilast™, everolimus™, Hirudin, steroids, anti-inflammatory agents such as ibuprofen, antimicrobials or antibiotics (e.g., Actinomycin D), tissue plasma activators, and agents that affect VSMC proliferation or migration such as transcription factor E2F1 (Goukassian et al., Circ. Res. 93(2): 162-169 (2003), which is hereby incorporated by reference in its entirety) or CD9 inhibitors (e.g., anti-CD9 antibodies such as mAb7 and CD9 fragments containing extracellular loop 2 (amino acids 168-192)), IL-10 inhibitors, and P13K inhibitors (e.g., LY294002 from Calbiochem (San Diego, Calif.)), CD40L antagonists, PARP1 inhibitor (e.g., PJ34 from Calbiochem) (Zhang et al., Am. J. Physiol. Heart Circ. Physiol. 287:H659666 (2004), which is hereby incorporated by reference in its entirety).

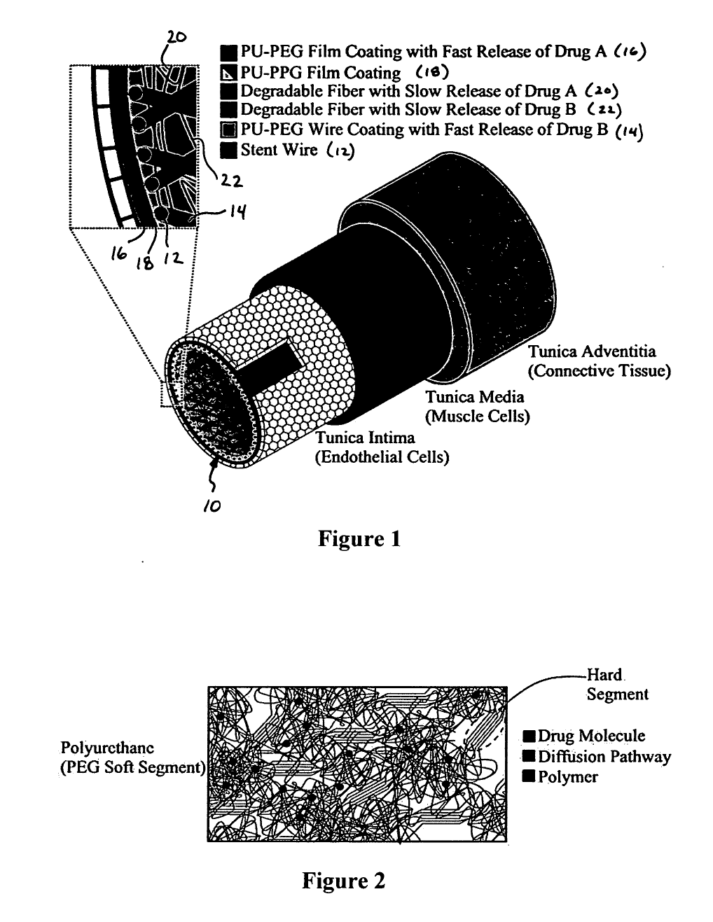

An exemplary vascular stent of the present invention is illustrated in FIG. 1. The stent includes an expandable mesh stent 12 (e.g., Palmaz-Schatz™) that is coated with a drug-eluting film 14 (i.e., a first layer) carrying an anti-thrombotic agent alone or in combination with an agent that promotes re-endothelialization. The coating 14 provides for fast drug release of one or both of these drugs. Two outer layers 16, 18 are provided. The outermost layer 16 is a drug-eluting film carrying an agent that promotes re-endothelialization (that is the same or different from the drug carried by the film 14), and the intermediate layer 18 is a polyurethane-polypropylene glycol film into which are embedded degradable drug-releasing fibers 20, 22. Fiber 20 is a single or bi-component fiber that carries an agent that promotes re-endothelialization for slow release. Fiber 22 is a single or bi-component fiber that carries an anti-thrombotic agent for slow release.

In this embodiment, the outermost layer 16 is a polyurethane-polyethylene glycol (PEG) matrix that includes VEGF. This material can be used for the outer stent coating to achieve rapid release of VEGF into endothelial cells of the tunica intima to encourage rapid re-endothelialization onto the inner stent surface. Slow release of VEGF by fibers 20 encourages re-endothelialization through the stent.

In this embodiment, the coating 14 is a polyurethane-PEG matrix that includes a GPVI antagonist. This material can be used to coat the stent metal with a thin film to achieve rapid and intense release of the GPVI antagonist to inhibit in-stent thrombosis, which usually occurs in an acute setting. Slow release of the GPVI antagonist to inhibit in-stent thrombosis over a long time period also can be achieved by placing this agent in fibers 22 that degrade slowly.

According to one embodiment, the outer layers 14, 16 are substantially impermeable to all cells (i.e., having an average pore width of up to a few micrometers and a pore shape which is highly elongated). According to another embodiment, the outer layers 14, 16 are porous to squamous epithelial or endothelial cells but not VSMC (i.e., having an average pore width of up to about 5 μm -10 μm and a pore shape which is highly elongated).

Depending upon the desired drug elution rate(s), the various polymeric layers (i.e., the first polymeric layer, the second polymeric layer, and the one or more additional polymeric layers) can be formed of different materials, including films, fibers, or combinations thereof. In general, films function as drug reservoirs to dispense larger amounts of drugs, and their microstructure can be engineered to achieve rapid release. Fibers can be used to achieve slower drug release during their biodegradation. Single component and bi-component fibers can be used, and the fibers can be embedded in films or present in woven or non-woven fabrics. Single component fibers can be produced from one polymer or co-polymers that degrade slowly and uniformly. Bi-component fibers can be produced as core-shell fibers so that one polymer contains a drug in the fiber shell, whereas another polymer contains the same or different drug in the fiber core. Fine sizes of bi-component fibers provide a large surface area that allows rapid delivery of drug from fiber shell, but slower delivery of drug from fiber cores. More coarse fibers provide slower release from shells and cores.

The use of textiles in biomedical applications has increased substantially with the advent of new fibers and technology. All biomedical textiles are formed from natural or synthetic fibers. These textiles are used in medical products and devices ranging from wound dressings to sophisticated devices such as vascular implants and tissue engineered scaffolds (King et al., Can. Textile J. 108(4):24-30 (1991), which is hereby incorporated by reference in its entirety). The biomedical applicability depends on the specific fiber configuration: monofilament or multifilament, twisted or braided, type of polymer-natural or synthetic, and performance-degradable or non-degradable. The textile fibers can be fibers in the nanoscale range or fibers having diameters in the range of up to several diamteres.

In the present invention, flexible drug elution can be achieved by any combination of up to three different techniques: (1) elution from a phase-separated polyurethane; (2) elution from the core and/or shell of a core-shell fiber; and (3) elution of a surface-grafted/ bonded drug molecule.

Elution from a phase-separated polyurethane allows for an initial drug delivery over the first week. Polyurethanes that have phase-separated morphology increase the life-time of drug release, due to the hard segment's interference in the diffusion pathway of the drug, as seen in FIG. 2 (Kim et al., Internat'l J. Pharm. 201:29-36 (2000), which is hereby incorporated by reference in its entirety). This is distinct of the diffusion profile afforded by traditional drug-eluting polymers, which will release drugs more quickly.

Drug-eluting fibers can be formed by any of a variety of approaches. Exemplary approaches including without limitation electrospinning, and bi-component fiber (BCF) techniques, and melt-blowing (MB).

The electrospinning (ES) process uses strong electrostatic forces to attenuate polymer solutions into solid fibers that have diameters in the range of 10-1000 nm. These fine fibers produce large surface-to-volume ratios that promise to provide new levels of performance for textile materials. The diameter of the nanofibers depends on the chemistry, viscosity, strength, and uniformity of the operating conditions. These nanofibers have been used to fabricate ultra-thin filter membranes, nonwoven mats for wound dressings, and scaffolds for tissue engineering.

ES polyurethane fibers with fiber diameter in the range of 500-600 nm have been prepared. Numerous other polymers including poly(ethylene oxide), polycarbonate, polystyrene, polyacrylonitrile, and polyamide have been successfully electrospun (Tsai et al., 16^{th} AFS Annual Technical Conference and Exposition, Jun. 17-20, 2003). ES has also been employed to produce nonwoven mats from Type I collagen and synthetic polymers, such as poly(lactide), poly(lactide-co-glycolide), poly(vinyl alcohol), poly(ethylene oxide), and poly(ethylene-co-vinyl acetate). Furthermore, a genetically engineered elastin-biomimetic peptide polymer has been electrospun (Ratner et al., Biomaterials Science 2ed. 89 (2004), which is hereby incorporated by reference in its entirety).

Although one of the major limitations of ES is the low production rate of single syringe-based polymer delivery, it is important to note that this problem is believed not to be a serious limitation for the present application considered here. That is because only a thin covering of fibers on a small object (stent) is needed. In the examples provided herein, it has been observed that a stent can be adequately coated with ES fibers in less than 5 minutes.

Bi-component fiber (BCF) technology, which typically consists of a core-shell configuration, has also been used for drug delivery. Hybrid BCF filaments may have a shell of a bioabsorbable polymer such as PLA or PGA, and a core of less bioabsorbable or nonabsorbable polymer such as PET. Alternatively, multifilament yarns may have bioabsorbable and nonabsorbable filaments lopped or braided together. This technology allows the healing process to be controlled by slowing the exposure of the nonabsorbable polymer (Ratner et al., Biomaterials Science 2ed. 91 (2004), which is hereby incorporated by reference in its entirety).

Bicomponent fibers having two or more polymer types (nylon and polyester, polypropylene and polyethylene, etc.) have been melt spun with configurations of core/sheath, side-by-side, or segmented pie for over 25 years (Zhao et al. J. Applied Polymer Science 85:2885-2889 (2002); Zhao et al., Polymer Engineering and Science 43(2):463-469 (2003); Zhao et al., Polymer International 52(1):133-137 (2003); Zhou et al., J. Applied Polymer Science 89:1145-1150 (2003), each of which is hereby incorporated by reference in its entirety).

The melt blowing (MB) process produces webs from thermoplastic polymers (Wente, Ind. Eng. Chem., 48:1342-1346 (1956), U.S. Pat. No. 3,972,759 to Buntin, U.S. Pat. No. 3,849,241 to Buntin et al., Wadsworth et al., INDA J. Nonwovens Res. 2(1):43-48 (1990), each of which is hereby incorporated by reference in its entirety). The MB process is compatible for use with bi-component fibers of the type described above. The most notable advantage of the single step MB process is its ability to produce webs at high speed that are composed of microfibers of about 1-9 μm diameter. The elasticity of MB PU webs allows for conformation of the stent to the wall of the vessel. This feature may be useful to achieve better adhesion between the mesh of the stent cage and the vessel.

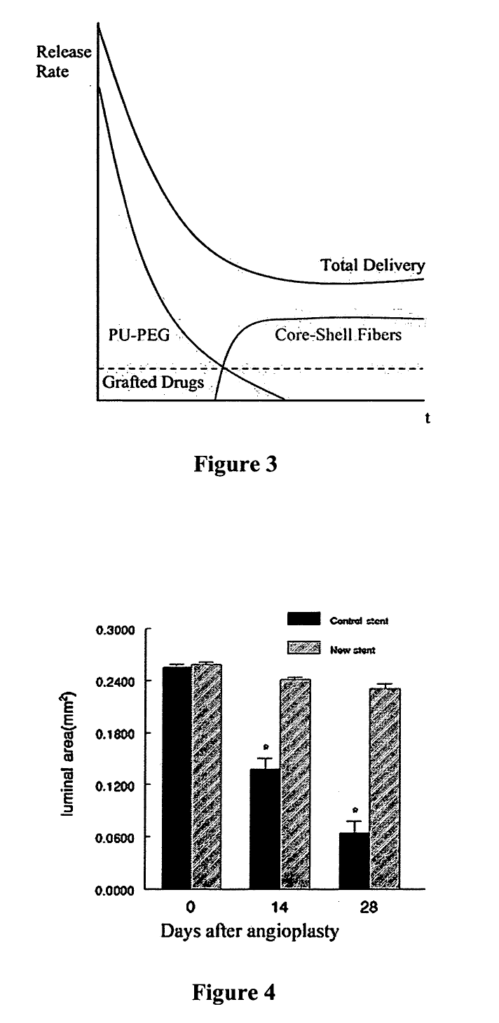

The BCF technique allows for delayed drug release because the drug is in the core of the fiber, and the shell must be degraded substantially before the drug can be eluted. Electrospinning can produce a distribution of fiber diameters, resulting in a release profile as shown in FIG. 3.

The third technique, surface-grafted/bonded drug, provides a constant low-level chemical signal attached to the coating of the stent by fibrin glue or grafting onto the polyurethane (FIG. 3).

The vascular stents of the present invention can be prepared using several processing steps.

In a first step, a first polymeric material can be applied to at least an internal surface of an expandable stent, thereby forming the first polymer layer exposed to the interior compartment of the stent. The first polymeric material includes the polymer components (as described above) and an agent that promotes re-endothelialization, an agent that inhibits thrombosis, or a combination thereof Curing of the polymeric material can be complete or partially complete before proceeding to subsequent steps.

According to preferred approaches, the expandable mesh stent is dip- or spray-coated with the bulk drug-polymer solution that will form the first polymeric layer. Dip-coating will coat entire mesh stent, not just the internal surface of the stent. Depending upon the manner of spray coating, spraying can cover primarily the internal surface or the entire stent.

In a second step, at least an outer surface of the expandable stent is covered with a second polymeric material in a manner that maintains stent expandability and forms a porous layer having pores that are substantially impermeable to vascular smooth muscle cell migration, thereby forming the second polymeric layer. To maintain expandability, the stent can be expanded prior to the covering step.

Exemplary procedures for the covering step include, without limitation, micro-extrusion of thermoplastic polymer filaments around the circumference of collapsed and balloon-expanded stents; electrostatic spinning (ES) of nanofibers around stents; encasement of stents in layers (i.e., composites) of fine filaments and nanofibers; and melt blown microfibers around stents. Any drugs incorporated into the fabric can be incorporated prior to fabrication of the stent covering.

Porosity of the second polymeric layer can be controlled during the covering procedure. Specifically, both pore size and pore shape can be controlled during processing. Pore size can be controlled by varying fiber diameter, web basis weight, and collector movement. Pore shape can be controlled by manipulating the die-to-collector distance (DCD) and primary airflow rate (Bresee et al., Internat'l Nonwovens J .13(1):49-55 (2004); Bresee et al., Internat'l Nonwovens J. 14(2):11-18 (2005). DCD adjustments and primary airflow rate control pore aspect ratio.

Any intermediate layers, i.e., between the expandable mesh stent and the second polymeric layer, can be applied prior to the covering with the second polymeric layer. As described in the preferred embodiment above, i.e., with a polymeric film embedded with polymer fibers, these materials can be applied by spraying, brushing, or roller coating the film onto the preceding layer of the stent.

In use, the stent will be inserted into a vessel of a patient using, e.g., a balloon catheter, to allow for expansion of the stent. Once expanded, the stent will be left in place as the instrument is withdrawn from the vessel, and surgical incisions closed. This is typically performed following angioplasty.

The patient is typically one who exhibits conditions associated with coronary artery disease, peripheral artery disease, or stroke, in which case medical intervention is warranted. Patients can be any animal, preferably mammals, most preferably humans, non-human primates, pigs, rabbits, horses, cows, sheep, llamas, or bison.

Prior to insertion of the stent, it is also possible to seed the interior surface of the first layer (i.e., the stent lumen) with endothelial cells, preferably endothelial cells harvested directly from the patient to be treated. Seeding of the stent can further promote re-endothelialization.

As a consequence of using stents of the present invention, the inventive stents can reduce in-stent thrombosis relative to conventional mesh stents and reduce in-stent neointimal hyperplasia and restenosis relative to conventional mesh stents (by substantially precluding migration of VSMC internally of stent). For these reasons, it is believed that the vascular stents of the present invention will afford higher success rates for vascular stents in the long-term treatment of coronary artery disease, peripheral artery disease, or stroke.

### EXAMPLES

The examples discussed below are intended to illustrate the present invention and are, by no means, intended to limit the claimed subject matter.

### Example 1

### Comparison of Conventional Stent to Stent Having Outer Polyethylene Layer Impermeable to Cells

Prototype barrier stents were prepared by Scientific Commodity, Inc., at the request of the inventors using an outer polyethylene layer that is impermeable to all cells. These prototype stents were compared in vivo to conventional mesh stents.

Rat carotid artery balloon angioplasty was performed as described in our previous study (Hamuro et al., J. Vasc. Interv. Radiol. 12(5):607-611 (2001), which is hereby incorporated by reference in its entirety). Immediately after angioplasty, the stents were implanted into the injured carotid arteries. The animals were sacrificed immediately after (0 day) and at 14 and 28 days after stent implantation, and the stented segments were isolated for histological analysis. As shown in FIG. 4, the luminal areas in carotid arteries with the prototype (new) stents are greater that those with conventional stents. These results suggest that use of a cell impermeable layer will increase luminal area after angioplasty.

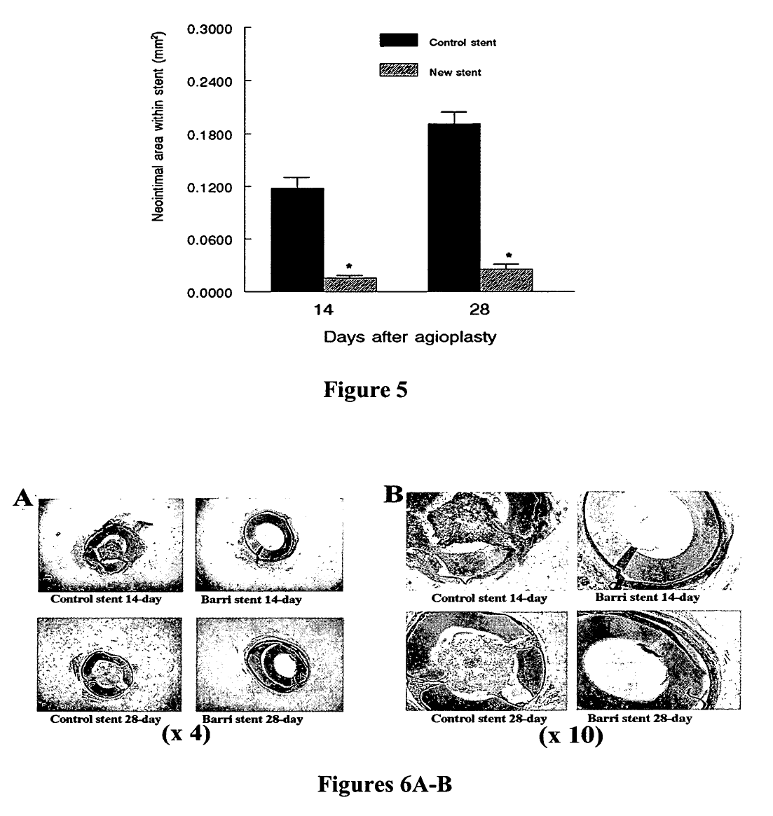

The neointima formation within the stents was then measured using an image analysis system. As shown in FIG. 5, the neointima formation within the prototype stent was significantly smaller than that within the conventional mesh stents. Therefore, the prototype stent that is cell impermeable decreases neointima formation within the stent after angioplasty.

FIGS. 6A-B illustrate representative photomicrographs of hematoxylin-eosin stained sections of rat carotid arteries from rats treated with the conventional mesh stents and prototype stents. There is only very small neointima formation within the prototype stent, whereas the neointima formation within the conventional stent is huge. Accordingly, the luminal area in carotid artery treated with the prototype stent is much greater that that treated with the conventional mesh stent (FIG. 4).

Together, these result suggest that prototype stents that are impermeable to VSMC cells may be useful in preventing or diminishing neointimal ingrowth and restenosis.

### Example 2

### Synthesis and Evaluation of Outer Coating Materials

The selection of polyurethanes for outer stent coatings is based on biocompatibility (Brown, J. Intraveneous Nursing 18:120-122 (1995); Szycher et al., Medical Devices Technol. 3:42-51 (1992); Jeschke et al., J. Vascular Srg. 29:168-176 (1999), each of which is hereby incorporated by reference in its entirety).

Polyurethanes are polymers consisting of hard and soft segments within the molecular chain. The morphology of polyurethane is characterized by the aggregation of hard segments, rigid domains, dispersed in a matrix of the soft segments. The phase separation is due to the chemical differences between the hard and soft segments. The polyurethane chemistry permits tailoring of properties to meet numerous applications through the appropriate selection of the reactive intermediates: diisocyanates, soft segment, and chain coupler. Polyurethane elastomers exhibit elastic behavior under low stress conditions. The more elastic behavior occurs when the concentration of hard segments is smaller, whereas plastic deformation is observed when hard segment concentration is large. Similarly, greater hardness and better stress resistance but lower resistance to abrasion is obtained when hard segment concentration is increased (Szycher et al., Medical Devices Technol. 3:42-51 (1992), which is hereby incorporated by reference in its entirety). For a given diisocyanate and coupler, the mechanical properties (Benson et al., J. Polymer Sci. Polymer Chem. 26:1393-1404 (1988), which is hereby incorporated by reference in its entirety) and hemocompatibility are directly related to molecular weight of the soft segment (Lyman et al., Trans. Amer. Soc. Artif. Inter. Organs 21:49 (1975), which is hereby incorporated by reference in its entirety). The polyurethanes used in biomedical applications are based on a polyether or polyester soft segment. Polyurethanes based on polyether soft segment are commonly used for implantable devices due to their hydrolytic stability.

A wide variety of polyurethane elastomers can be synthesized. For example, polyurethanes may be based on methylene diisocyanate (MDI), aliphatic compounds not related to MDI, polyether soft segments (polypropylene glycol (PPG), polytetramethylene glycol (PTMG), and polyethylene glycol (PEG)) and chain couplers (1,4-butanediol and ethylene diamine). Three soft segments with different molecular weights-2000, 1000, and 700-can be used in the synthesis to achieve materials with properties designed to vary through the desired range. Synthesis can be performed by the two-step polymerization method (Lyman, J. Polymer Sci. 45:49 (1960); Conjeevaram et al., J. Polymer Sci. Polymer Chem. 23:429-444 (1984), each of which is hereby incorporated by reference in its entirety).

PEG based polyurethanes are inherently more hydrophilic than most nonabsorbable polymer coatings. Continuous hydrophilic coatings based on waterbome polyurethanes can allow rapid diffusion of water through the membrane. To make them more hydrophilic, these coatings may incorporate up to 40% poly(ethylene glycol) (PEG).

The polyurethanes (PU) should be evaluated in terms of their processability and relevant mechanical, chemical, and barrier properties necessary for stent insertion and longevity after insertion. Mechanical testing will provide information regarding the tensile strength and strain-at-break. Additional testing such as abrasion and chemical resistance also can be performed on the various processed material forms-nonwovens, microfibers, nanofiber webs, and electrospun webs.

Polyurethane materials can be evaluated comprehensively for use as stent materials to promote desirable tissue growth, to facilitate blood flow, and to exhibit adequate durability. In addition to hemocompatibility, these materials also offer processing flexibility because they can be applied from an aqueous dispersion, from an organic solvent, or as a thermally extruded film, or as a fiber.

Meltblown Polyurethane Fabric Coating

Meltblown thermoplastic polyurethane (Noveon Estane 58245, a polyether TPU) microfibers were deposited on a scaled-up (12 mm) metal stent rotated by hand. The analysis of pore size and other characterizations of the stent fabrics was performed on the scaled-up 12 mm stent and on flat fabrics collected under as similar processing conditions as possible.

Process conditions included a die temperature from 425° F. (218° C.) to 450° F. (232° C.), hot air temperature from 450° F. (232° C.) to 500° F. (260° C.), a 60° angle nose tip with 25 spinneret holes per linear inch and hole diameters of 15 mils, a 30-mil die tip setback from the outer edge of each air knife, an air knife gap of 30 mils between the inside plane of each air knife and the nose tip, a polymer throughput rate of 0.2-0.4 g/hole/min, and a hot air flow rate of approximatelyl20 scfm/inch of die width. The MB fibers were collected at a distance of approximately 14 inches either onto the hand-rotated stent mandrel or onto a belt collector to produce flat web samples. The thickness of stent cover tubes was varied by rotating the stent mandrels for different amounts of time in the fiber stream. In commercial production, the distance of the rotating stent mandrel from the MB or ES die will be controlled by an electric precision drive system which maintains a constant specified surface speed, constant specified distance from the MB die and height in relation to the fiber stream being deposited on it.

Electrospun Stent Coverfabric

Polyurethane (Noveon Estane 58238) was electrospun from a syringe needle onto either a paper-coated flat collector or a scaled-up rotating metal stent. Noveon Estane 58238 is a polyester PU that may be either melt spun as a thermoplastic polyurethane or electrospun in a solvent. The electrospun solution that was prepared contained 15% 58238 PU/42.5% tetrahydrofuran (THF)/42.5% dimethylformamide (DMF). A DC voltage of 18 KV was applied through the clamp on the syringe needle, the collector was grounded and the distance between the end of the syringe needle and the flat collecting surface or rotating metal stent form was approximately 6 inches.

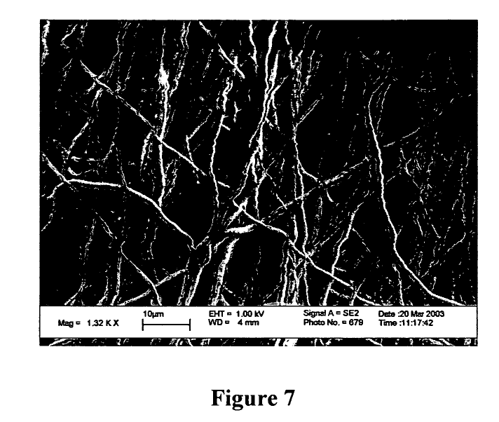

From previous experience, the diameters of ES TPU fibers are known to range from 100 to 600 nanometers. An exemplary image illustrating electrospun polyurethane is shown in FIG. 7.

To produce a fibrous cover on actual 3-6 mm stents, the actual expanded metal stents will be covered using either the meltblown or electrspun process. This will allow the elastic stent to be collapsed prior to vascular insertion, at which time the entire assembly can be expanded during angioplasty and vascular stenting. As an alternative to directly coating the stent, a replicate cage can be coated and then the stent covers removed; the cover can then be installed onto a vascular stent prior to its installation into the vessel of a patient.

Demonstration of Ability to Expand and Contract ES PU Stent Cover

An ES TPU coating was produced on a metal wire spring having an outside diameter of 5 mm and a length of 6 cm. Then, the fibrous tube was unrolled from the end of the stent form attached to the handle and pulled inside-out about 6 cm. A continuous thin covering of fibers remained on the wire when the tube was pulled out, indicating that the covering would remain adhered to the stent during contraction and later expansion of the stent. The removed tube was in a collapsed form, as it would be on a collapsed stent before the angioplasty procedure. Upon introducing a stream of pressurized fluid through the removed cover (by mouth), the cover expanded under influence of the pressure. The process was repeated several times with no apparent loss of elasticity or mechanical strength. This demonstrates the electrospun polyurethane covering materials can be expanded as they will be on a stent during use.

Thickness, Weight and Porosity of MB and ES Cover Fabrics

TPU 58245 was MB as flat fabric and as tubes on the scaled-up (12 mm) stent mandrel. Table 1 shows testing results from these webs. Although much thinner MB fabric and tubes can be produced, the flat fabrics had average thickness values 0.97 mm to 1.98 mm, with corresponding weights in grams per square meter (gsm) of 217 and 492 gsm, respectively. The average fiber diameters (as determined by computer assisted optical microscopy measurements) of the fabrics ranged from 3.8 to 5.4 micrometers (μm) and the corresponding mean pore sizes were 12.7 μm and 7.1 μm. It is interesting to note that Sample 2.1 MB had a lowest thickness of the flat fabrics at 0.97 mm, and still had a relatively low mean pore diameter of 10.0 μm, indicating that other factors such as fiber laydown, in addition to fiber diameters and small changes in MB conditions, can affect mean pore size. T. 1 MB and T.3 MB TPU stent tubes had average thickness values of 0.90 and 0.84 mm, with respective average weights of 115 and 138 gsm and respective average mean pore sizes of 7.8 and 6.2μm.

Table 1 also shows that ES flat fabrics had much thinner and lighter fabrics ranging from 0.031 to 0.160 mm with respective average weights of 9.8 and 7.1 gsm and respective mean pore sizes of 11.1 and 11.5 μm. It is quite notable that the thinnest and thickest ES flat fabrics had nearly the same mean pore size. As with MB, uniformity of fiber collection, fiber size and small processing changes afford the demonstrated means of controlling pore size while producing thin stent tubes.

The experimental ES PU stent tube Samples T. 1 and T.2 had very thin walls compared to MB tubes at 0.14 and 0.18 mm with respective weights of 35.1 and 28.3 gsm. Sample T.1 ES had a mean pore size of only 1.8 μm. Although this stent would allow small molecules to pass, it is expected to be impermeable to smooth muscle cells and endothelial cells.

TABLE 1
Melt blown (MB) and Electrospun (ES) Stent Cover Properties

| | | | | |
|---|---|---|---|---|
| | Thickness | Weight | Avg Fiber | Mean Pore |
| Sample No. | (mm) | (g)/(gsm) | D.(μm) | D.(μm) |

Estane 58245 Polyether TPU

| | MB TPU Flat un-wound Fabric | | | |
|---|---|---|---|---|
| 1.1 MB | 1.74 | 0.410/424 | 3.8 | 12.7 |
| 2.1 MB | 0.97 | 0.210/217 | 5.3 | 10.0 |
| 2.2 MB | 1.98 | 0.476/492 | 5.4 | 7.1 |
| | MB Experimental Stent Tubes (12 mm Dia) | | | |
| T.1 MB | 0.90 | 0.111/115 | 3.9 | 7.8 |
| T.3 MB | 0.84 | 0.134/138 | — | 6.2 |

Estane 58238 Polyester PU

| | ES PU Flat Fabric | | | |
|---|---|---|---|---|
| 1 ES | 0.040 | 0.0086/8.9 | — | 15.3 |
| 3 ES | 0.031 | 0.0095/9.8 | — | 11.1 |
| 2.2 ES | 0.072 | 0.0074/7.7 | — | 10.0 |
| 2.3 ES | 0.160 | 0.0069/7.1 | — | 11.5 |
| | ES Experimental Stent Tubes (5 mm Dia) | | | |
| T.1 ES | 0.14 | 0.034/35.1 | — | 1.8 |
| T.2 ES | 0.18 | 0.028/28.9 | — | — |

### Prospective Example 3—

### Synthesis and Evaluation of Mixed Fiber/Film Coating Materials

A composite fibrous polyurethane material using appropriate layers of continuous filament microfibers, nonwoven webs of microfibers, and nonwoven webs of nanofibers will be synthesized. Continuous filaments will be produced using micro-extrusion melt spinning (MS) techniques, nonwoven webs made of microfibers will be produced using melt blowing (MB), and nonwoven webs made of nanofibers will be produced using electrospinning (ES). The polyurethanes that will be used in ES do not need to be melt processable since the polymer is dissolved in solvent.

Continuous filaments of PU will be produced first using a micro-extruder with an air quench, drawing and continuous take-up system (e.g., Randcastle Microtruder Model No. RCPR with a ⅝-inch diameter screw, single spinneret die, two godets for drawing the extruded filaments). Extruded filaments will be unwound and tested for biocompatibility, degradation, and mechanical properties.

Optimized PU filaments will be hand-wound around large ½-inch to 2-inch stent replicas (either obtained from the stent manufacturer or custom-built). Fatigue properties will be studied after 1, 5, and 20 cycles from the collapsed to balloon-expanded states. Hand-wound stent replicas will be examined by optical microscopy to access structural changes on macro and micro levels. Single filaments will be removed and tested for tensile strength and elongation-to-break, and compared to control filaments before the cycle test to help evaluate durability of the extruded filaments. Tensile and elastic recovery measurements (e.g., using United Tensile Tester Model No. SSTM-1-E-PC) also will help determine whether filaments have the proper mechanical properties and will guide PU modification or replacement. Since the surface texture of the filaments may change during stent collapse/expansion, fibers also will be examined by scanning electron microscopy.

Prototype stents for in vivo use will be wound on a high-speed winder, which will provide automated winding of filaments with greater control. Macro and micro level structural changes of stents/replicas will be accessed by electron and optical microscopy. The contact angle and wetting characteristics of whole stents will be determined (e.g., using a Kruss DSA100 Expert System) before and after different collapse-expansion cycles. The strength, elongation to break, and surface texture of single fibers will be evaluated again after 1, 5 and 20 collapse-to-expansion cycles of the stents/replicas formed by automated winding.

After completing analysis of the optimal PU filament, similar measurements will be acquired for microfibers formed by melt blowing and nanofibers formed by electrospinning. Composite materials produced by a combination of melt spun single filaments, webs of microfibers formed by melt blowing, and webs of nanofibers formed by electrospinning will also be manufactured and tested.

In the same manner as described above, composite coatings of single and multiple fibers deposited on a PU film by ES and MB will be prepared to study drug delivery and stent durability properties.

### Prospective Example 4

### In vitro Testing of Coated Stents

Both stents with non-permeable coatings and selectively permeable coatings will be assessed. Blood permeability of the coated stent will be tested using an in vitro perfusion system (Swanson et al., Int. J. Cardiol. 92(2-3):247-251 (2003), which is hereby incorporated by reference in its entirety). The stent segment of the circuit will be immersed into a glass collection chamber containing PBS. The perfusate will be heparinized-rabbit blood. The perfusion pressure will be kept at the physiologic level and the flow rate will be initially maintained at 10 mL/min using a peristaltic pump (Watson-Marlow 302S). Sterile silicone tubing (3-mm bore, Fisons) will be used to carry the perfusate to the chamber housing. Different conditions will be used to examine stent permeability that mimic normal and pathologic (stenosed coronary arteries) blood flow. After 1,2, 4, 6, and 12-hour perfusion, the solution outside of the glass chamber will be collected to measure for the presence of blood cells via Coulter counter analyses and for protein levels by the BioRad protein determination assay. The inside of the stent will be examined by microscopy to examine for blood cell adhesion and any bound protein eluted with 0.1% SDS detergent. Protein levels will also be assessed by the Bio Rad method. Selective permeability to desirable cells, such as squamous epithelial cells, under physiological pressure will be assessed via microscopy.

Both VEGF and GPVI antagonist release kinetics will also be assessed in vitro as previously described (Palmerini et al., J. Am. Coll. Cardiol. 44(8):1570-1577 (2004), which is hereby incorporated by reference in its entirety). In this experiment, ¹²⁵I-labeled VEGF or ¹²⁵I-labeled GPVI antagonist will be coated into inner layer of the stent via dip-coating or spray-coating. The radiolabeled stent will then immersed in an in vitro perfusion circuit as described above (Swanson et al., Int. J. Cardiol. 92(2-3):247-251 (2003); Palmerini et al., J. Am. Coll. Cardiol. 44(8):1570-1577 (2004), each of which is hereby incorporated by reference in its entirety) and will be perfused continuously at 10 mL/min in the closed-loop circuit with PBS containing 1% BSA. VEGF or GPVI release will be counted in a gamma well counter. Totally, six ¹²⁵I-labeled VEGF-coated inventive stents and ¹²⁵I-labeled GPVI antagonist will be needed for the experiment. The perfusing solution will be changed routinely every 4 hours for a 48 hr period to determine kinetics of elution. For extended studies, an HPLC detection method may be implemented due to the short half-life of the radioisotope.

The complete or selective blockage of migration of VSMC, endothelial cells, fibroblasts, and leukocytes will be assessed for one or more of the nonwoven elastic coatings. In this experiment, human aortic endothelial cells, human aortic smooth muscle cells, human HL-60 cells and human fibroblast cell line MRC-5 will be used. Endothelial cells will be trypsinized and subcultured in culture medium (MCDB-131; Sigma), supplemented with fibroblast growth factor, epidermal growth factor, hydrocortisone, and penicillin/streptomycin containing 10% bovine calf iron supplemented serum at 37° C. in a 5% CO₂ incubator. The culture medium will be exchanged every 48 hours. Human aortic VSMC will be obtained from Clonetics and cultured in recommended culture medium (SmGM-2, Clonetics). Media will be replaced every other day. The cultured VSMC will be used between passages 4 and 7. Human leukemia (HL-60) cells will be obtained from American Type Culture Collection and grown in RPMI 1640 medium supplemented with 10% heat-inactivated fetal bovine serum, 100 units/ml penicillin-streptomycin, and 2 mM L-glutamine. Me₂SO (1.3% v/v) will be added to the cells for 7 days to induce differentiation to a neutrophilic phenotype. For fibroblast cell line MRC-5, the cells will be grown and maintained as monolayers in Minimal Essential Medium (Gibco BRL), supplemented with 10% heat-inactivated fetal calf serum, 50 IU/ml of penicillin and 50 μg/ml of streptomycin sulfate at 37° C. in a 5% CO₂ atmosphere.

Cell migration assays will be performed using modified Boyden chambers (Transwell-Costar Corp.) with and without stent segments (impermeable to cells and selectively permeable) coated on the underside with 10 μg/ml fibronectin. Subconfluent cells will be trypsinized (0.01% trypsin/5 mM EDTA; Cambrex), neutralized (Cascade Biologics, Inc.), washed with EBM/0.1% BSA, and resuspended. Typically, 5×10⁵ cells will be added to the top of each migration chamber and allowed to migrate to the underside of the test material for 4-24 h. Cells will be fixed and stained (Hema 3 Stain System; Fisher Diagnostics). The number of migrated cells per membrane will be captured using bright-field microscopy connected to a Spot digital camera (Diagnostic Instruments). Migrated cells from the captured image will be counted using NIH Image software.

The extent and rate of endothelial cell growth (endothelialization) in the stent inner layer will be assessed in an endothelial cell culture system. Human aortic endothelial cells will be obtained from Clonetics and used between passages 4 and 10. Cells will be cultured as described above.

The effect of VEGF-coated stents on endothelial cell growth (endothelialization) in the inner surface of stents will be measured using an in vitro cell migration assay reported recently (Palmerini et al., J. Am. Coll. Cardiol. 44(8):1570-1577 (2004); Baron et al., Cardiovasc. Res. 46(3):585-594 (2000), each of which is hereby incorporated by reference in its entirety). Briefly, to simulate arterial wall surface, firm fibrin gel will be prepared as follows: fibrinogen (Sigma) dissolved in phosphate buffered saline (1.5 mg/mL) will be adjusted to pH 7.2 with 0.1 mol/L HCl. This fibrinogen solution will then be poured into 100-mm×100-mm Petri dishes and spread evenly immediately after initiating polymerization by adding thrombin (Sigma) to a final concentration of 0.625 U/mL of fibrinogen solution. The gels will be rinsed four times with phosphate buffered saline and incubated overnight in culture medium at 37° C. in a 5% CO₂ incubator. After removing the medium, human aortic endothelial cells will be seeded onto the gels at a density of 20,000 cells/cm² and cultured until a confluent layer of cells are attained (1-2 d). The confluence of cultured cells will be determined by visual (microscopic) inspection.

Three different stents (Control Palmaz-Schatz™ stent, inventive stent without VEGF coating and inventive stent with VEGF coating) will be pressed flat on the surface of the endothelialized gel in each dish. Before the placement of the stents, the cells in the area of the stent placement will be removed by scratch. The gels with the stents will be incubated at 37° C. in a 5% CO₂ incubator for 4, 7, 10 and 14 days to monitor endothelial cell migration onto the stents.

At 4, 7, 10 and 14 days, the stents will be rinsed with phosphate buffered saline, fixed in methanol for 5 minutes, and stained with 2% Giemsa stain. After staining, the distance of cell migration and the density of cells over each stent will be measured with use of reflective light microscopy. The distance of cell migration will be measured on a perpendicular line from the midpoint of each modified edge to the leading edge of advancing cells. Cell density on the metal surface will be determined as the number of cells per 100×field and expressed as cells/cm² . Every time point should contain six stents for every group.

Finally, the effect of inventive stents coated with GPVI antagonist will be assessed for platelet deposition and thrombosis in vitro. The antagonistic, agonistic, or anti-thrombotic activities of candidate compounds, including GPVI specific antibodies, antibody fragments, GPVI polypeptides, including soluble polypeptides, can be further assayed using the systems developed by Diaz-Ricart et al., Arteriosclerosis, Thromb. Vasc. Biol. 16:883-888 (1996), which is hereby incorporated by reference in its entirety. This assay determines the effect of candidate compounds on platelets under flow conditions using de-endotheliailized rabbit aorta and human endothelial cell matrices.

Platelet deposition and thrombosis on the control and inventive stents in vitro will also be measured using flow circuits as described previously (Fraker et al., Biochem. Biophys. Res. Commun. 80(4):849-857 (1978); Inoue et al., Atherosclerosis 162(2):345-353 (2002), each of which is hereby incorporated by reference in its entirety). Briefly, blood samples (30 ml) will be collected in a syringe containing 10 IU of heparin from rabbits. The platelets will be labeled with ¹¹¹⁻indium (¹¹¹In) or ⁵¹Cr using a standard technique (Zhang et al., Chin. Med. J (Engl.) 117(2):258-263 (2004), which is hereby incorporated by reference in its entirety). The radiolabelled platelets will be added to a further 100 ml of blood containing heparin (10 u ml⁻¹). Control Palmaz-Schatz™ stent and inventive stents coated with GPVI will be inserted and then deployed in silicone tubing (3 mm inner diameter) by inflating the balloon at 14 atm for 20 s. The silicone tubing will be then connected to a perfusion circuit which is set to pump the blood containing the ¹¹¹In-labelled platelets as perfusate at a flow rate of 10 ml/min, with a theoretically calculated shear rate of ≈64 s⁻¹ up to 1500s⁻¹. The circuit will then be closed using a silicone connector and the perfusion performed for 120 min. The temperature will be kept stable at 37° C. by a water bath. The stents will be rinsed and the radioactivity associated with each stent will be counted and quantified in a gamma counter (Packard Cobra series Auto-gamma counting system, 15-75 keV window). For some samples, the test material will be fixed and the adherent platelets will be examined microscopically for adhesion, spreading and the formation of filopodia that would indicate that not only did platelets adhere, but they also underwent an activation response. The material will be examined and scored for the presence, if any, of platelet aggregates.

Once candidate GPVI-inhibitory compounds are identified, the in vivo activity of these antagonists can be assayed using standard models of platelet function as described in Coller et al., Blood 66:1456-59 (1985); Coller et al., Blood 68:783-86 (1986); Coller et al., Circulation 80:1766-74 (1989); Coller et at al., Ann. Intern. Med. 109:635-38 (1988); Gold et al., Circulation 77:670-677 (1988); and Mickelson et al., J. Molec. Cell Cardiol. 21 :393-405 (1989), each of which is hereby incorporated by reference in its entirety.

### Prospective Example 5

### In vivo Testing of Coated Stents

Angioplasty will be performed in rabbit left carotid arteries followed by stent implantation with either an inventive stent or control Palmaz-SchatzTM stent.

New Zealand white rabbits (Myrtles Rabbitry, Thompson Station, Tenn, Male, 2.5-3.0 kg) will be used for the study. Carotid artery balloon angioplasty and stent implantation will be performed as described (Zhang et al., J. Biol. Chem. 276(29):27159-27165 (2001); Danenberg et al., Circulation 108(22):2798-2804 (2003), each of which is hereby incorporated by reference in its entirety). Animals will be anesthetized with an intramuscular injection of ketamine (35 mg/kg) and xylazine (5 mg/kg). After exposing the left common, external and internal carotid artery with their side branches, a sheath will be inserted in the first branch of the left external carotid artery. A 3F Fogarty catheter (Baxter Edwards) will be introduced through the sheath and advanced to the proximal edge of the omohyoid muscle. To produce carotid artery injury, we will inflate the balloon with saline and withdraw it 3 times from just under the proximal edge of the omohyoid muscle to the carotid bifurcation. After injury, Heparin (500 units) will be given. No anti-platelet agents or additional anticoagulants will be administered. The stent, either inventive stent (totally or selectively impermeable) or control Palmaz-SchatzTM stent, will be mechanically crimped on 3.0-mm-diameter, 20-mm-long balloon catheters (Johnson & Johnson) and inserted through the sheath into the injured common carotid artery. The balloon will be inflated to 10 atm for 60 seconds and then deflated (balloon/artery diameter ratio ≈(1.2-1.3): 1). The catheter will then be removed and the surgical wound will be closed.

The rabbits will be sacrificed at 7, 14, 28, 90 and 180 days after stent implantation. Before scarification, a Doppler flow probe (Transonic Systems, Inc.) will be inserted around the left stented common carotid artery and right uninjured common carotid artery and the blood flow will be measured as described previously (Van Belle et al., Circulation 95(2):438-448 (1997), which is hereby incorporated by reference in its entirety).

Neointimal formation within and outside the stents and luminal areas will be determined by histology. Briefly, after blood flow measurement, the arteries will be perfusion-fixed with 10% neutral buffered formalin at physiological pressure. Stented arteries will be isolated and embedded with a methacrylate formulation. Multiple sections 5 μm thick will be cut with a tungsten carbide knife (Delaware Diamond Knives) on an automated microtome (Leica, Inc) from the proximal and distal ends and the midpoint of each stented segment (Walter et al., Circulation 11 0(l):36-45 (2004), which is hereby incorporated by reference in its entirety). The sections will be stained with Verhoeff's elastin stain. Neointimal areas within and out side stent, and luminal area will be measured on Verhoeff's tissue elastin-stained sections via a computerized image analysis system (Scion Image CMS-800). As an initial study, only one time point (28 days) will be used to evaluate the benefit effect of the new stent.

To determine the effect of the inventive stent on re-endothelialization in rabbit carotid artery after angioplasty, rabbit carotid artery balloon injury and stent implantation will be performed as described above. Te animals will be will be sacrificed at 3, 7, 14 and 28 days after stent implantation. Re-endothelialization will be determined by scanning electron microscopy (Zhang et al., Arterioscler. Thromb. Vasc. Biol. 25(3):533-538 (2005); Zhang et al., J. Exp. Med. 199(6):763-774 (2004), each of which is hereby incorporated by reference in its entirety). Before scarification, animals will receive heparin (2000 U) via the ear vein. A cannula will insert into the left ventricle to perfuse in situ 100 mL of 5% dextrose solution with 100 U/mL heparin, followed by 0.25% silver nitrate for 20 seconds. This will be followed by 5% dextrose and then pressure-perfusion at 100 mm Hg for 2 hours with 10% buffered formalin. The stented carotid arteries will be isolated and cut longitudinally to open. Surface endothelialization will be quantified via a scanning electron microscopy equipped with 2× to 10×objectives and a pair of l Ox eyepieces. The visual field of the microscope can be integrated into the LED-lit cursor of a standard digitizing pad through a drawing tube attachment with an x1.25 magnification factor. Measurements will be carried out with (Scion Image CMS-800). Integration of the microscope with the computer via the digitizing tablet facilitated direct examination of the endothelial surface at x25 to x125.

To determine the effect of the inventive stents on in-stent thrombosis in rabbit carotid artery after angioplasty, rabbit carotid artery balloon injury and stent implantation will be performed as described above. Animals will be sacrificed at 1, 3, 7, 14 and 28 days after stent implantation to determine the in-stent thrombosis. Before scarification, animals will receive heparin (2000 U) via the ear vein. The stented carotid arteries will be perfused, isolated, cut as described above. Some vessels will be embedded with a methacrylate formulation and the cross sections will be cut for H-E staining. The in-stent thrombosis will be detected by histology analysis and the scanning electron microscopy (Zhang et al., Arterioscler. Thromb. Vasc. Biol. 25(3):533-538 (2005), which is hereby incorporated by reference in its entirety).

To determine the histological characteristics of neointima in the inventive stent and the long-term the biocompatibility of the inventive stent, the following immunohsitochemistry experiments will be performed. The rabbits will be sacrificed at 14, 28, 90 and 180 days after stent implantation. Before scarification, the arteries will be perfusion-fixed with 10% neutral buffered formalin in vivo at physiological pressure. After the perfusion, the stented carotid arteries will be isolated, embedded as described above. Immunostaining of VSMC, leukocyte, and endothelial cell will be performed in vessel cross sections (5 μM) using ABC kit (Vector Laboratories) as described previously (Hamuro et al., J. Vasc. Interv. Radiol. 12(5):607-611 (2001); Foo et al., Thromb. Haemost. 83(3):496-502 (2000); Aggarwal et al., Circulation 94(12):3311-3317 (1996), each of which is hereby incorporated by reference in its entirety). Prior to incubation with the primary antibody for 1 h, tissue sections will be treated with H₂O₂ to quench endogenous peroxide activity. A biotinylated secondary antibody will then be applied. Immunostaining will be detected using a Vector ABC kit. Control stains lacking primary or secondary antibodies will be performed. For leukocyte staining, mouse anti-rat CD45 (leukocyte common antigen, clone OX-1) (BD Pharmingen) will be used. For VSMC and endothelial cell, antibodies for the SMC biomarker, α-actin (Sigma), and the endothelial cell biomarker, von Willebrand factor (Dako), will be used followed by the standard indirect immunoperoxidase procedures. In addition, platelet and thrombosis will also be determined as described above.

The proposed experiments should allow us to test the effect of the final designed inventive stent on restenosis. Based on the pathological mechanism and the preliminary data presented herein, it is expected that the novel endovascular device will increase re-endothelialization, reduce thrombosis and reduce in-stent restenosis in the animal model, and any neointima within the inventive stent (whether totally or selectively impermeable) will have less VSMC. It is also expected that inventive stents will have a good long-term biocompatibility in vivo.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A vascular stent comprising:
an expandable stent defining an interior compartment;
a first polymeric layer exposed to the interior compartment defined by the stent, the first layer comprising an agent that promotes re-endothelialization, an agent that inhibits thrombosis, or a combination thereof; and
a second polymeric layer at least partially external of the stent, the second layer being adapted for contacting a vascular surface and being characterized by pores that are substantially impermeable to vascular smooth muscle cell migration.

2. The vascular stent according to claim 1 wherein the second layer is permeable to squamous epithelial cells or endothelial cells.

3. The vascular stent according to claim 1 wherein the first and second layers are independently formed of a polymer or co-polymers selected from the group consisting of polyurethane, poly(ethylene oxide), polycarbonate, polystyrene, polyacrylonitrile, polyamide, polyetherester, ethylene copolymers, polyesters, copolyesters, polyamides, polypropylene, polyethylene, or combinations thereof.

4. The vascular stent according to claim 1 wherein the second layer comprises a polyurethane-polyethylene glycol matrix.

5. The vascular stent according to claim 4 wherein the second layer further comprises an agent that promotes re-endothelialization or an anti-proliferative agent.

6. The vascular stent according to claim 5 wherein the an agent that promotes re-endothelialization is vascular endothelial growth factor (VEGF), angiopoietin 1, or avp3 agonists.

7. The vascular stent according to claim 5 wherein the anti-proliferative agent is transcription factor E2F1, a CD9 inhibitor, an IL-10 inhibitor, a P13K inhibitor, CD40L inhibitors, PARP1 inhibitor.

8. The vascular stent according to claim 5 wherein the polyurethane-polyethylene glycol matrix is characterized by the presence of channels that allow for diffusion from the second layer of the agent that promotes re-endothelialization and/or the anti-proliferative agent.

9. The vascular stent according to claim 1 further comprising:
a first and second drug-eluting fibers in a layer intermediate the second layer and the expandable stent.

10. The vascular stent according to claim 9 wherein the first fiber comprises an agent that inhibits thrombosis and the second fiber comprises an agent that promotes re-endothelialization.

11. The vascular stent according to claim 9 wherein the first and second fibers are each independently selected from the group consisting of single-component and bi-component fibers.

12. The vascular stent according to claim 1 wherein the first layer substantially encapsulates the stent.

13. The vascular stent according to claim 1 wherein the first layer comprises a polyurethane-polyethylene glycol matrix.

14. The vascular stent according to claim 13 wherein the polyurethane-polyethylene glycol matrix is characterized by the presence of channels that allow for diffusion from the first layer of the agent that promotes re-endothelialization and/or the agent that inhibits thrombosis.

15. The vascular stent according to claim 13 wherein the first layer comprises a GPVI antagonist, VEGF, or a combination thereof.

16. The vascular stent according to claim 1 wherein one or both of the first and second layers have adhered or grafted thereon an agent that promotes re-endothelialization, an agent that inhibits thrombosis, or a combination thereof.

17. The vascular stent according to claim 1 further comprising a drug selected from the group of basic fibroblast growth factor (bFGF) and active fragments thereof, rapamycin and rapamycin analogs, Taxol™ or Taxan™, antisense dexamethasone, angiopeptin, Batimistat™, Translast™, Halofuginon™, nicotine, acetylsalicylic acid, Tranilast™, everolimustm, Hirudin, steroids, ibuprofen, antimicrobials or antibiotics (e.g., Actinomycin D), tissue plasma activators, antifibrosis agents.

18. The vascular stent according to claim 1 wherein both the first and second layers comprise a polyurethane-polyethylene glycol matrix.

19. The vascular stent according to claim 1 wherein the pores of the second layer have an average width between about 100 nm up to about 5 μm.

20. The vascular stent according to claim 1 wherein the pores of the second layer have an average width between about 5 μm up to about 15 μm.

21. The vascular stent according to claim 1 wherein the pores of the second layer have a shape that is substantially elongated with an average pore aspect ratio between about 1.5 and about 20.

22. The vascular stent according to claim 1 wherein the second polymeric layer is in the form of a woven or non-woven fabric.

23. A method of preventing neointimal hyperplasia in a patient following insertion of a prosthetic graft, the method comprising:
providing a vascular stent according to claim 1; and
inserting the vascular stent at a vascular site of the patient, wherein the material of the second layer substantially precludes migration of VSMC internally of stent and the thereby prevents neointimal hyperplasia.

24. A method of preventing in-stent thrombosis, the method comprising:
providing a vascular stent according to claim 1;
inserting the vascular stent at a vascular site of the patient, wherein the first layer comprises an agent that inhibits thrombosis; and inserting the vascular stent at a vascular site of the patient, wherein release of the agent that inhibits thrombosis from the first layer substantially precludes aggregation of platelets and thereby prevents in-stent thrombosis.

25. A method of treating a coronary artery disease, peripheral artery disease, stroke, or other vascular bed disease, the method comprising:
providing a vascular stent according to claim 1;
performing angioplasty at a vascular site in a patient exhibiting conditions associated with coronary artery disease, peripheral artery disease, or stroke;
inserting the vascular stent at the vascular site, wherein said inserting substantially precludes neointima and in-stent thrombosis while promoting re-endothelialization, thereby treating coronary artery disease, peripheral artery disease, stroke, or other vascular bed disease.

26. A method of making a vascular stent comprising:
providing an expandable stent that defines an interior compartment;
applying to at least an internal surface of the expandable stent a first polymeric material comprising an agent that promotes re-endothelialization, an agent that inhibits thrombosis, or a combination thereof, thereby forming the first polymer layer exposed to the interior compartment;
covering at least an outer surface of the expandable stent with a second polymeric material in a manner that maintains stent expandability and forms a porous second polymeric layer having pores that are substantially impermeable to vascular smooth muscle cell migration.

27. The method according to claim 26 wherein said covering is carried out by micro-extrusion of thermoplastic polymer filaments around the stent, electrostatic spinning of nanofibers around the stent, encasement of the stent in layers of fine filaments and nanofibers, and melt blowing microfibers around stents.

28. The method according to claim 27 wherein said covering is carried out only externally of the stent.

29. The method according to claim 26 wherein said applying is carried out by spraying, dipping, brushing, or rolling.
